(19) United States
(12) Patent Application Publication
(10) Pub. No.: US 2011/0105972 A1
(43) Pub. Date: May 5, 2011

(54) BELT FOR POSTURE CORRECTION

(76) Inventor: Sang Hak Kim, Gyeonggi-do (KR)

(21) Appl. No.: 12/996,574

(22) PCT Filed: May 15, 2009
(86) PCT No.: PCT/KR2009/002586

§ 371 Date: Dec. 6, 2010

(30) Foreign Application Priority Data
Jun. 5, 2008 (KR) 20-2008-0007536

(51) Int. Cl. A61F 5/00

(52) U.S. Cl. 602/19

(57) ABSTRACT

The present invention is a belt for posture correction which has a separate thigh support portion such that the belt does not shift upwards, and which distributes stress concentrated at the knees while pressing the lumbar region by means of the tensile strength of the belt enclosing the waist and the knees. The present invention comprises a waist support part further comprising a waist pressing pad positioned at the lumbar region of the user, a first belt fixed to said waist pressing pad, connecting rings formed at both ends of said first belt; a pair of knee support parts which include a knee support pad positioned at the knee region to support the belt, a second belt fixed to said knee support pad, and a second belt length adjustment part for adjusting length of said second belt; a pair of thigh support parts which include a third belt connected vertically to the lower side of said second belt, a thigh support pad positioned at the thigh region and fixed to the third belt, and a third belt length adjustment part for adjusting the length of said third belt; and an interval adjustment part that includes two fourth belts extending toward the inside of the leg of the user from each of the above-mentioned left and right second belts, a fourth belt fastening means for fastening the two fourth belts formed on the left and right sides, and a fourth belt length adjustment part for adjusting the lengths of the fourth belts.

### TECHNICAL FIELD

The present invention relates to a belt for correcting posture, and in particular to a wearable belt for correcting posture which can prevent a user's vertebra from being bent in a forward or lateral direction when a user sits for thereby obtaining a right sitting posture.

### BACKGROUND ART

A vertebra is a key element which keeps a body balance in the course of various activities while supporting a body weight and protecting central nerves which are connected from a brain to each part of the body. When the vertebra bends due to a wrong posture in a forward or backward or lateral direction, it is impossible to keep a right body balance while pressing central nerves, which results in causing various pains.

A hip joint is a very important joint which connects legs and pelvis and helps a person walk in a right posture. So, it is needed to properly keep the position and angle of a hip joint for a normal walking posture. When a person sits on a chair for a long time, a ligament and muscle supporting a hip joint tend to be loosened by themselves, so the hip joint might have eversion phenomenon.

When the above abnormal posture keeps long, the eversion hip joint receives over loads, so the pelvis might be widened, which leads to causing various diseases such as an abnormal skeleton deformation and growth, scoliosis, waist disk, sciatica, etc.

The problems due to hip joints or vertebra can be cured by means of a surgical operation or an aiding tool. In this case, the conventional methods cost a lot and use a complicated mechanism which does not satisfy an easier posture correction demand during normal activities.

In order to overcome the above problems, a plurality of posture correction members are disclosed, most of which are focused on adjusting postures in a state that a waist and knees are connected using a belt when a user sits on a chair or a floor. The belt for adjusting vertebrae lumbales discloses the above features (Korean application number: 20-1998-0001858).

As shown in FIG. 1, the conventional posture correcting belt comprises a waist support part 1, a knee support part 2 and a belt 3, with a user's waist being pressed and supported using a belt which surrounds knees and waist.

However, the conventional posture correcting belt has a problem that a tensional force is focused on the user's knees when surrounding a belt on a waist and knees, so it is impossible for a user to keep a right posture.

In addition, the knee support part 2 supported by the knees when in use might escape. In this case, the user's posture becomes unbalanced, and the belt is needed to be fitted again. Since the user is forced to wear the belt again and again, the user feels uncomfortable and uneasy.

### DISCLOSURE OF INVENTION

Accordingly, it is an object of the present invention to provide a posture correcting belt which overcomes the problems encountered in the conventional art and comprises another belt which disperses stresses and is connected with a user's thigh portion for thereby preventing a knee support part supported by knees from escaping upward.

To achieve the above objects, there is provided a belt for correcting is posture which comprises a waist support part which includes a waist pressing pad provided at a user's vertebrae lumbales portion for pressing, a first belt fixedly connected with the waist pressing pad and a connection ring formed at both ends of the first belt; a pair of leg support parts which each includes a knee support pad provided at a knee portion of a user for supporting the belt, a second belt fixedly connected with the knee support pad and a second belt length adjusting part for adjusting the length of the second belt; a pair of thigh support parts which each includes a third belt vertically connected from a lower side of the second belt, a thigh support pad positioned at a thigh portion of a user and fixedly connected with the third belt, and a third belt length adjusting part for adjusting the length of the third belt; and an interval adjusting part which includes a fourth belt extended from the left and right second belts to an inner direction of the user's legs, a fourth belt engaging member for fixedly engaging the fourth belts of the left and right sides, and a fourth belt length adjusting part for adjusting the length of the fourth belt.

Preferably, the first belt further includes a one-touch engaging member for allowing the waist support part to be easily attached and detached.

Preferably, there is further provided a fixing belt for allowing the waist support part to closely fit a user's waist, said fixing belt including a fifth belt for surrounding a user's waist portion, a fifth belt engaging member formed at both ends of the fifth belt for fixedly engaging the same with each other, and a fifth belt length adjusting part for adjusting the length of the fifth belt.

### Effects

The posture correcting belt according to the present invention has the following advantages.

First, a belt connected surrounding user's knees and waist serves to press a user's vertebrae lumbales for thereby correcting postures.

Second, a thigh support part provided at a lower side serves to disperse the stress focused on user's knees while stably supporting.

Third, a thigh support part provided at a lower side serves to prevent a belt from escaping upward, with the belt surrounding user's knees and waist.

Fourth, a fixing belt surrounding a user's waist serves to allow a waist support part to tightly fit with a user.

### BRIEF DESCRIPTION OF THE DRAWINGS

The present invention will become better understood with reference to to the accompanying drawings which are given only by way of illustration and thus are not limitative of the present invention, wherein;

FIG. 1 is a side view illustrating a use state of a conventional posture correcting belt;

FIG. 2 is a perspective view illustrating a posture correcting belt is according to the present invention;

FIG. 3 is a perspective view illustrating a use state of a posture correcting belt according to the present invention;

FIG. 4 is a side view illustrating a use state of a posture correcting belt according to the present invention; and

FIG. 5 is a partially enlarged view illustrating a third belt length adjusting part of a posture correcting belt according to various examples of the present invention.

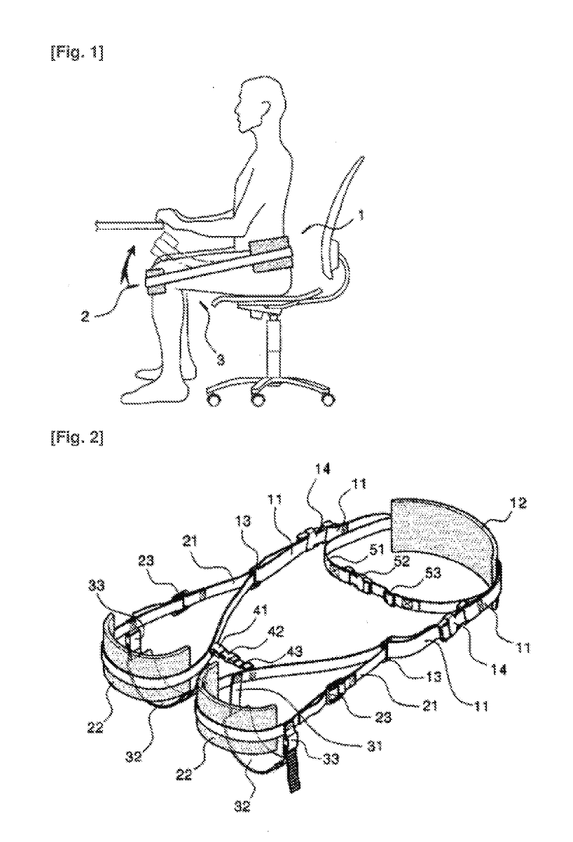

### BRIEF DESCRIPTIONS OF REFERENCE NUMERALS OF THE DRAWINGS

10: waist support part 11: first belt

12: waist pressing pad 13: connection ring

14: one-touch engaging member 20: knee support part

21: second belt 22: knee support pad

23: second belt length adjusting part

30: thigh support part

31: third belt 32: thigh support pad

33: third belt length adjusting part 40: interval adjusting part

41: fourth belt 42: fourth belt engaging member

43: fourth belt length adjusting part 50: fixing belt

51: fifth belt 52: fifth belt engaging member

53: fifth belt length adjusting part

### MODES FOR CARRYING OUT THE INVENTION

The present invention will be described with reference to the accompanying drawings.

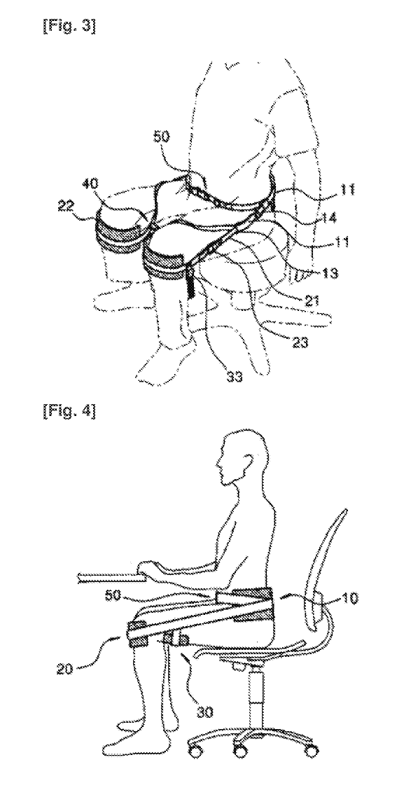

FIG. 2 is a perspective view illustrating a posture correcting belt according to the present invention, and FIG. 3 is a perspective view illustrating a use state of a posture correcting belt according to the present invention, and FIG. 4 is a side view illustrating a use state of a posture correcting belt according to the present invention.

As shown in FIGS. 2 to 4, the present invention comprises a belt type thigh support part which presses a vertebrae lumbales by means of a tensional force of a belt by connecting a vertebrae lumbales portion of a waist and knees for thereby correcting a posture and disperses a stress focused on a knee side and prevents a belt from escaping upward, with the belt type thigh support part being supported by a thigh side.

The present invention is basically directed to providing a posture correcting belt which presses and supports a vertebrae lumbales in a state that a user sits. The posture correcting belt according to the present invention comprises a waist support part 10, a knee support part 20, a thigh support part 30, an interval adjusting pat 40 and a fixing belt 50.

The waist support part 10 comprises a waist pressing pad 12 disposed at a vertebrae lumbales for pressing, a first belt 11 fixedly connected with the waist pressing pad 12, and a connection ring 13 formed at both ends of the first belt 11. A one-touch engaging member 14 is further provided at a certain portion of the first belt 11 for easily attaching or detaching the waist support part 10.

The first belt 11 surrounds the waist and vertebrae lumbales of a user with both ends being extended toward user's knees. The connection ring 13 formed at both ends is connected with the second belt 21 of the knee support part 20.

Preferably, the waist support part 10 can be formed in various forms depending on a user's demand. The waist pressing pad 12 might be to formed in various forms by using acupressure protrusions, and a unique effect can be obtained depending on the shape of a waist pressing pad 12.

The one-touch engaging member 14 formed at the first belt 11 allows the waist support part 10 to be easily attached or detached while providing a convenient function when changing the waist support part 10 and is maintaining and correcting the posture.

The knee support part 20 comprises a knee support pad 22 provided at a user's knee portion for supporting a belt, a second belt 21 fixedly connected with the knee support pad 22, and a second belt length adjusting part 23 for adjusting the length of the second belt 21.

The knee support part 20 is provided in one pair for the use at left and right knees. The second belt 21 which surrounds user's knees and is extended to the side of a waist passes through the connection ring 13 of the waist support part 10. At this time, the knee support pad 22 is provided at the second belt 21 and a portion contacting with the user's knees.

The first belt 11 and the second belt 21 are connected between the waist pressing pad 12 and the knee support pad 22. The user's knees and waist vertebrae lumbales are pressed with the aid of the first and second belts 11 and 21 for thereby correcting a wrong posture.

The thigh support part 30 is provided to disperse a tensional force which is generally focused on the knee support part 20 and to prevent the first and second belts 11 and 21, which connect the knees and waist, from escaping upward. The thigh support pat 30 comprises a third belt 31 vertically connected toward the lower side of the second belt 21, a thigh support pad 32 provided at a thigh portion of a user's body and fixedly connected with the third belt 31, and a third belt length adjusting part 33 for adjusting the length of the third belt 31.

The thigh support part 30 is provided in one pair for connecting to the knee support parts 20 of the left and right sides. The third belt 31 surrounds the lower sides of the user's thighs and is connected, and the thigh support pad 32 is provided between the third belt 31 and the user's thighs.

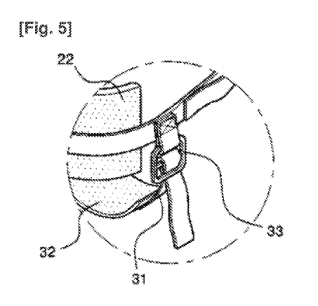

The third belt length adjusting part 33 can be formed in various forms. The third belt length adjusting part of FIG. 2 and the third belt length adjusting part 33 of FIG. 5 can be selectively applied depending on the embodiments. Since the constructions of the third belt length adjusting parts 3 are known in the art, the descriptions of the same will be omitted.

The interval adjusting part 40 is provided to adjust the interval between the left and right legs and comprises a fourth belt 41 extended from the left and right second belts 21 to the inner direction of the user's legs, a fourth belt engaging member 42 for fixing the left and right fourth belts 41, and a fourth belt length adjusting part 43 for adjusting the length of the fourth belt 41.

The fixing belt 50 is provided to allow the waist support part 10 to closely contact with the user's waist and comprises a fifth belt 51 for surrounding the user's waist, a fifth belt engaging member 52 formed at both ends of the fifth belt 51 for fixedly engaging the same, and a fifth belt length adjusting part 53 for adjusting the length of the fifth belt 51. The fifth belt 51 is fixedly connected at the waist pressing pad 12 along with the first belt 11.

In addition, the fixing belt 50 is provided a comfortable and easy posture in the course of posture correction and allows the waist support part 10 to closely fit the user's waist even when the first belt 11 is disengaged by using the one-touch engaging member 14.

The posture correcting belt according to an embodiment of the present invention is basically directed to pulling and pressing a user's waist vertebrae lumbales with the aid of the construction formed of a first belt 11 surrounding a user's waist and an extended second belt 21 surrounding user's knees for thereby correcting a user's wrong posture. The ring shaped third belt 31 is provided at a lower side of the second belt 21. The third belt 31 surrounding the lower sides of the thighs disperses the tensional force, which is generally focused on the knees, to the thighs and prevents the first and second belts 11 and 21 from escaping in the upward direction of the thighs. The fifth belt 51 surrounds the user's waist, and the waist support pat 10 is fixedly connected, so the user can sit in a stable posture.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A belt for correcting posture, comprising:
a waist support part 10 which includes a waist pressing pad 12 provided at a user's vertebrae lumbales portion for pressing, a first belt 11 fixedly connected with the waist pressing pad 12 and a connection ring 13 formed at both ends of the first belt 11;
a pair of leg support parts 20 which each includes a knee support pad 22 provided at a knee portion of a user for supporting the belt, a second belt 21 fixedly connected with the knee support pad 22 and a second belt 21 length adjusting part 23 for adjusting the length of the second belt;
a pair of thigh support parts 30 which each includes a third belt 31 vertically connected from a lower side of the second belt 21, a thigh support pad 32 positioned at a thigh portion of a user and fixedly connected with the third belt 31, and a third belt length adjusting part 33 for adjusting the length of the third belt 31; and
an interval adjusting part 40 which includes a fourth belt 41 extended from the left and right second belts 21 to an inner direction of the user's legs, a fourth belt engaging means 42 for fixedly engaging the fourth belts 41 of the left and right sides, and a fourth belt 41 length adjusting part 43 for adjusting the length of the fourth belt 41.

2. A belt for correcting posture according to claim 1, wherein said first belt 11 further includes a one-touch engaging means 14 for allowing the waist support part 10 to be easily attached and detached.

3. A belt for correcting posture according to claim 1, further comprising a fixing belt 50 for allowing the waist support part 10 to closely fit a user's waist, said fixing belt 50 including a fifth belt 51 for surrounding a user's waist portion, a fifth belt engaging means 52 formed at both ends of the fifth belt 51 for fixedly engaging the same with each other, and a fifth belt length adjusting part 53 for adjusting the length of the fifth belt 51.
